(19) United States
(12) Patent Application Publication
(10) Pub. No.: US 2009/0041814 A1
(43) Pub. Date: Feb. 12, 2009

(54) COSMETIC COMPOSITION

(75) Inventor: Teruyuki NANBU, Hakata-ku (JP)

Correspondence Address: OBLON, SPIVAK, MCCLELLAND MAIER & NEUSTADT, P.C., 1940 DUKE STREET, ALEXANDRIA, VA, 22314, US

(73) Assignee: ASKA CORPORATION Co. Ltd., Hakata-ku (JP)

(21) Appl. No.: 12/051,991

(22) Filed: Mar. 20, 2008

(30) Foreign Application Priority Data
Aug. 7, 2007 (JP) 2007-205030

(51) Int. Cl. A61K 8/02; A61K 31/715; A61Q 99/00

(52) U.S. Cl. 424/401; 514/54

(57) ABSTRACT

The invention provides a cosmetic composition which can smooth fine lines and wrinkles, impart firmness and elasticity to the facial skin, and make fine lines and wrinkles less noticeable, all within a short period of time. The cosmetic composition is in a hydrogel form and contains sodium hyaluronate, modified cornstarch, and pullulan, particularly in amounts of 0.1 to 1.0 wt. %, 0.5 to 5.0 wt. %, and 0.1 to 3.0 wt. %, respectively. The composition may further contain a dispersant.

### BACKGROUND OF THE INVENTION

1. Field of the Invention

The present invention relates to a cosmetic composition, and more particularly to a hydrogel cosmetic composition for smoothing fine lines and wrinkles on the face and restoring tonicity and elasticity in the facial skin.

2. Background Art

There have conventionally been proposed various cosmetic products for the eye area, which contain hyaluronic acid or a salt thereof (sodium salt), or a hyaluronic acid derivative (see, for example, Japanese Patent Application Laid-Open (kokai) Nos. 2007-70330 and 2005-68073). Those conventional cosmetic products enhance permeability of sodium hyaluronate through the skin, to thereby suppress formation of fine lines and wrinkles in the skin, to provide the skin area around the eyes with firmness and elasticity, or to prevent or reduce wrinkles, sagging, and similar problems of the skin.

However, the aforementioned conventional cosmetic products fail to satisfactorily meet the consumers' requirements that, within only a short period of time, fine lines and wrinkles are smoothed or made less noticeable, and firmness and elasticity of the facial skin is restored.

### SUMMARY OF THE INVENTION

In view of the foregoing, an object of the present invention is to provide a cosmetic composition which, within a short period of time, can smooth fine lines and wrinkles, impart firmness and elasticity to the facial skin, and make fine lines and wrinkles less noticeable.

Accordingly, the present invention provides a hydrogel cosmetic composition containing sodium hyaluronate, modified cornstarch, and pullulan.

The cosmetic composition of the present invention develops, within only a short period of time, the effects of smoothing fine lines and wrinkles, imparting firmness and elasticity to the facial skin, and making fine lines and wrinkles less noticeable.

### DETAILED DESCRIPTION OF PREFERRED EMBODIMENTS

The cosmetic composition of the present invention contains sodium hyaluronate, modified cornstarch, and pullulan. Since the cosmetic composition contains these three components in combination, as demonstrated in the Examples hereinbelow, the composition exhibits remarkable effects which cannot be attained by cosmetic compositions containing only one of these components.

Preferably, the cosmetic composition of the present invention contains sodium hyaluronate in an amount of 0.1 to 1.0 wt. %, modified cornstarch in an amount of 0.5 to 5.0 wt. %, and pullulan in an amount of 0.1 to 3.0 wt. %.

When the cosmetic composition of the present invention contains these three components in amounts falling within the above ranges, the object of the present invention can be more effectively achieved. Most preferably, the cosmetic composition of the present invention contains sodium hyaluronate in an amount of 0.5 wt. %, modified cornstarch in an amount of 2.5 wt. %, and pullulan in an amount of 0.5 wt. %.

Generally, the cosmetic composition of the present invention may contain a dispersant so as to sufficiently disperse the aforementioned three components in water. Examples of the dispersant which may be employed include 1,3-butylene glycol, glycerin, diglycerin, propylene glycol, and dipropylene glycol. These dispersants may be employed singly or in combination of two or more species. The cosmetic composition of the present invention may contain such a dispersant in an amount of 1.0 to 30.0 wt. %.

The cosmetic composition of the present invention may be prepared through the following procedure: sodium hyaluronate (powder) is sufficiently mixed with a dispersant to thereby form a mixture; modified cornstarch and pullulan are added to the mixture, followed by stirring for mixing; heated water (e.g., 40° C. to 80° C.) is added to the resultant mixture, followed by sufficient stirring by means of a mixer; and the resultant mixture is cooled. Through cooling, a gel composition is obtained.

When the cosmetic composition of the present invention is applied to the eye area, within several minutes after application of the composition the user can actually sense and realize that the skin of the eye area has firmness and elasticity. When the cosmetic composition of the present invention is continuously applied to the face, fine lines and wrinkles can be reduced. Therefore, the cosmetic composition of the present invention is particularly suitable for use as a cosmetic product for the eye area.

### EXAMPLES

The present invention will next be described by way of examples, which should not be construed as limiting the invention thereto.

### Comparative Example 1

Sodium hyaluronate powder was added to a dispersant (1,3-butylene glycol), followed by stirring. The resultant dispersion was added to water which had been heated in advance to 60° C., and the mixture was stirred by means of a mixer for about 30 minutes, followed by cooling to room temperature, to thereby yield a cosmetic composition. The formulation of the cosmetic composition is shown in Table 1.

### Comparative Example 2

The procedure of Comparative Example 1 was repeated, except that sodium hyaluronate powder was substituted by modified cornstarch, to thereby yield a cosmetic composition. The formulation of the cosmetic composition is shown in Table 1.

### Comparative Example 3

The procedure of Comparative Example 1 was repeated, except that sodium hyaluronate powder was substituted by pullulan, to thereby yield a cosmetic composition. The formulation of the cosmetic composition is shown in Table 1.

### Example 1

Sodium hyaluronate (powder) was sufficiently mixed with a dispersant (1,3-butylene glycol), and modified cornstarch and pullulan were added to and mixed with the resultant mixture, followed by stirring. The resultant dispersion was added to water which had been heated in advance to 60° C., and the mixture was sufficiently stirred by means of a mixer, followed by cooling to room temperature, to thereby yield a gel cosmetic composition. The formulation of the cosmetic composition is shown in Table 1.

TABLE 1

| | Comparative | Comparative | Comparative | |
|---|---|---|---|---|
| Components | Example 1 | Example 2 | Example 3 | Example 1 |
| Sodium | 0.5 wt. % | — | — | 0.5 wt. % |
| hyaluronate | | | | |
| Modified | — | 0.2 wt. % | — | 0.2 wt. % |
| cornstarch | | | | |
| Pullulan | — | — | 0.5 wt. % | 0.5 wt. % |
| Dispersant | 15 wt. % | 15 wt. % | 15 wt. % | 15 wt. % |
| Water | Balance | Balance | Balance | Balance |
| Total | 100 wt. % | 100 wt. % | 100 wt. % | 100 wt. % |

### <Evaluation of Cosmetic Composition in Terms of Firmness & Elasticity>

The cosmetic compositions prepared in Comparative Examples 1 to 3 and Example 1 were evaluated by 15 female panelists who asserted themselves as having fine lines and wrinkles around the eyes. Specifically, each panelist applied the cosmetic compositions to their eye area and evaluated 30 minutes after application thereof in terms of firmness and elasticity of the composition-applied area according to the following four criteria: (1) no improvement in terms of firmness and elasticity, (2) slight improvement in terms of firmness and elasticity, (3) some improvement in terms of firmness and elasticity, and (4) substantial improvement in terms of firmness and elasticity. Table 2 shows the numbers of panelists who gave the corresponding evaluations.

TABLE 2

| | (1) | (2) | (3) | (4) |
|---|---|---|---|---|
| | No | Slight | Some | Substantial |
| | improvement | improvement | improvement | improvement |
| | in firmness | in firmness | in firmness | in firmness & |
| | & elasticity | & elasticity | & elasticity | elasticity |
| Comparative | 1 | 2 | 9 | 3 |
| Example 1 | | | | |
| Comparative | 9 | 5 | 1 | 0 |
| Example 2 | | | | |
| Comparative | 13 | 2 | 0 | 0 |
| Example 3 | | | | |
| Example 1 | 0 | 1 | 2 | 12 |

### <Evaluation of Cosmetic Composition in Terms of Effect on Fine Lines and Wrinkles>

The cosmetic compositions prepared in Comparative Examples 1 to 3 and Example 1 were evaluated by 15 female panelists who asserted themselves as having fine lines and wrinkles around the eyes. Specifically, each panelist applied the cosmetic composition to their eye area once a day for one month, and evaluated in terms of effect on fine lines and wrinkles at the composition-applied area according to the following four criteria: (1) fine lines and wrinkles have increased in number or depth, (2) no change, (3) fine lines and wrinkles have been slightly smoothed, and (4) fine lines and wrinkles have been clearly smoothed. Table 3 shows the numbers of panelists who gave the corresponding evaluations.

TABLE 3

| | (1) | | (3) | (4) |
|---|---|---|---|---|
| | Increased or | (2) | Slightly | Clearly |
| | deepened lines | No | smoothed lines | smoothed lines |
| | and wrinkles | change | and wrinkles | and wrinkles |
| Comparative | 1 | 2 | 9 | 3 |
| Example 1 | | | | |
| Comparative | 9 | 5 | 1 | 0 |
| Example 2 | | | | |
| Comparative | 13 | 2 | 0 | 0 |
| Example 3 | | | | |
| Example 1 | 0 | 1 | 2 | 12 |

As is clear from the above data, the cosmetic composition of the present invention, which contains sodium hyaluronate, modified cornstarch, and pullulan in combination, exhibits remarkably excellent effects, as compared with cosmetic compositions containing only one of these three components.

The present invention finds wide utility in the cosmetic industry, and is also applicable to chemical products.

What is claimed is:

1. A cosmetic composition in the form of hydrogel, the composition comprising sodium hyaluronate, modified cornstarch, and pullulan.

2. A cosmetic composition as described in claim 1, which contains sodium hyaluronate in an amount of 0.1 to 1.0 wt. %, modified cornstarch in an amount of 0.5 to 5.0 wt. %, and pullulan in an amount of 0.1 to 3.0 wt. %.

3. A cosmetic composition as described in claim 1 or 2, which further contains a dispersant.

4. A cosmetic composition as described in claim 3, which contains a dispersant in an amount of 1.0 to 30.0 wt. %.

5. A cosmetic composition as described in claim 4, wherein the dispersant is selected from the group consisting of 1,3-butylene glycol, glycerin, diglycerin, propylene glycol, and dipropylene glycol.
